(19) United States
(12) Patent Application Publication
(10) Pub. No.: US 2026/0345492 A1
(43) Pub. Date: Nov. 5, 2026

(54) CATION TRIGGERED LIPOSOMES

(71) Applicant: Robert WOODBURN, III, Ogden Dunes, IN (US)

(72) Inventor: Robert WOODBURN, III, Ogden Dunes, IN (US)

(21) Appl. No.: 19/106,043

(22) PCT Filed: Aug. 25, 2023
(86) PCT No.: PCT/US2023/072906

§ 371 Date: Feb. 24, 2025

Related U.S. Application Data
(60) Provisional application No. 63/400,881, filed on Aug. 25, 2022.

(51) Int. Cl. A61K 9/1272; A61K 41/00; A61K 47/02; A61K 47/18; A61K 47/24; A61K 47/28

(52) U.S. Cl. CPC A61K 9/1272; A61K 41/0042; A61K 47/02; A61K 47/183; A61K 47/24; A61K 47/28

(57) ABSTRACT

The present disclosure is directed to liposomes and their use in delivery and release of drugs upon exposure to ionizing radiation. More particularly, the liposomes of the present invention comprise a cation triggered membrane permeabilizing lipid that is activated in the presence of free cations, wherein the liposome further comprises an agent that releases free cations upon excitation with ionizing radiation.

### CROSS-REFERENCE TO RELATED APPLICATIONS

This application claims priority under 35 U.S.C. § 119(e) to U.S. Provisional Application No. 63/400,881, filed Aug. 25, 2022, which is expressly incorporated by reference herein.

### BACKGROUND

Liposomes are self-assembled vesicles having a spherical bilayer structure surrounding an aqueous core domain. Liposomes can range in size from about 20 and about 30,000 nm in diameter and can be unlaminated or multilaminate (i.e., comprising concentric lipid bilayers). Typically, liposomes can be divided into three categories based on their overall size and the nature of the lamellar structure. The three classifications, as developed by the New York Academy Sciences Meeting, “Liposomes and Their Use in Biology and Medicine,” of December, 1977, are multi-lamellar vesicles (MLV's), small uni-lamellar vesicles (SUV's) and large uni-lamellar vesicles (LUV's).

Liposome have been previously disclosed for use as delivery vehicles that are capable of carrying both hydrophobic cargo in the lipid bilayer and/or hydrophilic cargo in the aqueous core. Liposome size is usually in a range from about 50 to about 250 nm, which is particularly suitable for targeted delivery of chemotherapy agents to solid tumor sites via the enhanced permeability and retention of cancer tissues (the EPR effect) (Maeda, H., et al., J. Controlled Release. 65(1-2): 271 (2000)). The preferential accumulation of drug-containing liposomes at the tumor site via EPR provides a means for localizing the drug, improving drug efficacy, and reducing drug toxicity to normal cells or tissues. For example, Doxil™, an FDA-approved liposome product containing doxorubicin, has been shown to have reduced toxicity compared with the free drug (Martin, F. J., et al., “Clinical pharmacology and antitumor efficacy of DOXIL.” Medical Applications of Liposomes. Ed. D. D. Lasic. Amsterdam: Elsevier, 1998, pp 635-688).

However, the benefits of liposomal drug delivery vehicles are limited by drawbacks including liposome metabolism and excretion from the body. In particular, optimizing the release rate of a liposomal drug is a difficult balancing act between in vivo half life and release. In general, leaky liposomes will make the encapsulated drug more available, but cause more risk in toxicity similar to the free drug. On the other hand, less leaky liposomes may reduce toxicity, but they may not provide the desirable drug release for efficacy. Thus while liposomes are already used for cancer treatment, the efficacy of such treatments is hampered by the lack of an effective mechanism to trigger the release of the incorporated drug.

Various triggering mechanisms have been proposed to release liposome content upon delivery to a target site. These mechanisms include temperature triggered release (US 20070264322), use of nano particles (US 20130028962) use of trigger polypeptides (US 20060210549) and the use of photopolymerization. An extensive review of methods to photochemically reorganize lipid bilayers has been published (O'Brien et al., Bioorganic Photochemistry 1993, 2:111-167).

However, there is still a need for a liposome delivery vehicle that has sufficient stability to allow for effective delivery to a target site while having a triggering mechanism that effectively releases the liposomal content at the target site.

### SUMMARY

The present disclosure is directed to the use of membrane permeabilizing lipids that are triggered by contact with cations to shift to a conformation that destabilizes an otherwise stable lipid bilayer. More particularly, in one embodiment a liposome is provided comprising a lipid bilayer that defines an interior luminal space of the liposome, wherein the lipid bilayer comprises a cation triggered membrane permeabilizing lipid. The cation induced conformational change of the membrane permeabilizing lipids present in the lipid bilayer of the liposome produces macromolecule-sized defects in lipid bilayers, resulting in the release of the liposome contents. In one embodiment the cation-triggered liposomes of the present disclosure comprise a cation responsive membrane permeabilizing lipid, and an inducible source of free cations. Optionally, the cation-triggered liposomes further comprise a therapeutic agent entrapped within the lipid membrane or contained in the liposome luminal space.

Advantageously, the cation responsive membrane permeabilizing lipids disclosed herein will only induce defects in a lipid bilayer after exposure to a source of free cations. Accordingly, the cation-triggered liposomes comprising the cation responsive membrane permeabilizing lipids remain stable until the membrane permeabilizing lipids are contacted with cations. Contact of the membrane permeabilizing lipids with cations induces a conformational change in the membrane permeabilizing lipids that disrupts membrane integrity of the liposome and results in the rapid release of liposome contents. In one embodiment, the cation-triggered liposomes of the present disclosure comprise a cation (such as Ca²⁺) that is initially sequestered by a chelating agent/molecular cage wherein the chelating agent/molecular cage is designed to release bound cations upon exposure to radiation, including for example exposure to UV rays or X rays, gamma rays, neutrons or protons. In one embodiment the chelating agent/molecular cage is designed to release bound cations upon exposure to X rays.

In one embodiment the cation-triggered liposomes disclosed herein comprise a source of cations, wherein the cations are sequestered by a photolabile molecular cage, such that the cation is released in response to exposure to light. For example light in the ultraviolet (UV) to infrared wavelength range may mediate release of the cation from the molecular cage. In one embodiment the molecular cage for releasably sequestering a cation (including for example Ca²⁺) is Bis(acetoxymethyl) 3,12-bis(2-(acetoxymethoxy)-2-oxoethyl)-5-(4,5-dimethoxy-2-nitrophenyl)-6,9-dioxa-3,12-diazatetradecane-1,14-dioate (DMNPE-4 AM-caged-calcium):

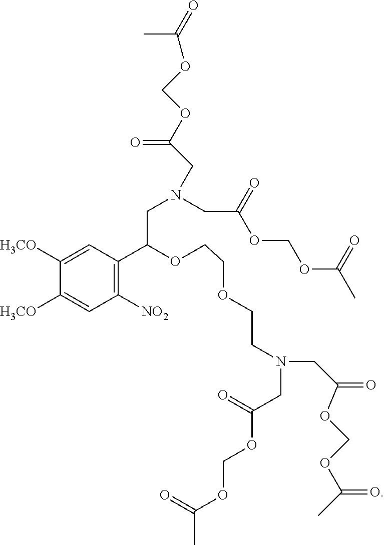 [SMILES: COc1cc(C(CN(CC(=O)OCOC(C)=O)CC(=O)OCOC(C)=O)OCCOCCN(CC(=O)OCOC(C)=O)CC(=O)OCOC(C)=O)c([N+](=O)[O-])cc1OC]

In another embodiment the molecular cage for releasably sequestering a cation (including for example Ca²⁺) is DM-Nitrophen™ (Calbiochem):

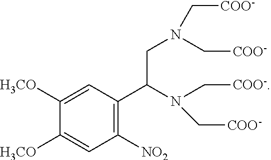

Thus upon exposure of the molecular cage to UV light, cations are released within the lumen of the cation-triggered liposome, and the free cations then interact with the membrane permeabilizing lipids to induce a conformation change in the cation responsive membrane permeabilizing lipids to induce the formation of macromolecule-sized defects in lipid bilayers and cause a liposome to release its contents.

In one embodiment, the cation-triggered liposome also contains a nanoscintillator that is responsive to radiation. The radiation source that induces the nanoscintillators to emit UV light may be external to the patient administered the cation-triggered liposomes, or may be internal to the patient in the form of radioactive seeds, ribbons, or capsules. In one embodiment, the target is exposed to radiation, such as X-rays, to cause the nanoscintillators to emit UV light. The emitted UV light induces the release of cations from the chelating agent/molecular cage, resulting in their binding to the cation responsive membrane permeabilizing lipids, inducing the membrane permeabilizing lipids to undergo a conformational change that disrupts membrane integrity of the liposome and releases the liposome contents. In one embodiment the nanoscintillator is LuPO₄.

The cation-triggered liposomes of the present disclosure further comprise a stable liposome-forming lipid component comprising one or more standard lipids known to those skilled in the art for preparing liposomes. For example lipids known to form stable liposomes include, but are not limited to, phosphatidylcholine (PC), phosphatidylethanolamine (PE), phosphatidic acid (PA), phosphatidylglycerol (PG), phospatidylserine PS) and phosphatidylinositol (PI), and non-natural lipid(s) and cation lipid(s) such as DOTMA (N-(1-(2,3-dioxyloxy)propyl)-N,N,N-trimethyl ammonium chloride) and 1,2-distearoyl-sn-glycero-3-phosphocholine (DSPC). The cation responsive membrane permeabilizing lipids are present at a relatively low percentage relative to the total lipid content of the liposome membrane. In one embodiment the ratio of stable standard liposome-forming lipid to cation responsive membrane permeabilizing lipids is selected from ratios from about 20:1 to about 10:1. In one embodiment the cation responsive membrane permeabilizing lipids are present at 5, 6, 7, 8, 9, 10 or 20% relative to the total phospholipds comprising the liposome membrane.

In one embodiment the cation responsive membrane permeabilizing lipid is a lipid that is conformationally changed upon binding Ca²⁺, wherein the conformation induced by binding Ca²⁺ disrupts the liposomal membrane and increases the rate of release of therapeutic compounds entrapped by the liposome. In one embodiment the cation permeabilizing lipid has the general structure of

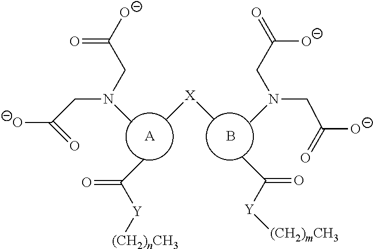

wherein rings A and B are independently 5 or 6 membered cycloalkyl, aryl or heteroaryl rings;
X is a linker of 3 to 6 atoms selected from C₁-C₆ alkyl, C₁-C₅ heteroalkyl, and —O(CH₂)_{z}O—, where z is an integer selected from 1 to 4;
Y is NH or CH₂; and
n and m are independently an integer selected from the range of 9-17. In one embodiment the cation permeabilizing lipid has the general structure of

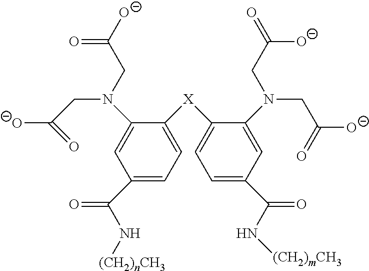

wherein X is a linker of 3 to 6 atoms selected from C₁-C₆ alkyl, C₁-C₅ heteroalkyl, and —O(CH₂)_{z}O—, where z is an integer selected from 1 to 4, optionally wherein X is —O(CH₂)_{z}O—; and
n and m are independently an integer selected from the range of 9-17, optionally wherein n and m are the same.

In one embodiment the cation permeabilizing lipid has the general structure of compound 1:

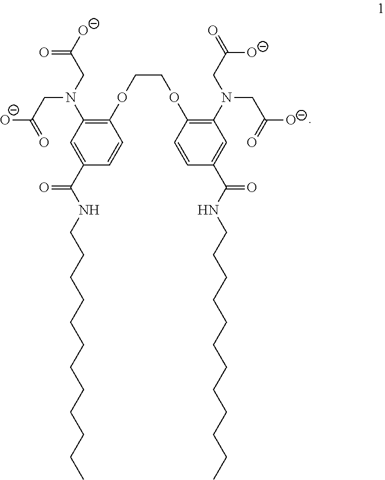 [SMILES: CCCCCCCCCCCCNC(=O)c1ccc(OCCOc2ccc(C(=O)NCCCCCCCCCCCC)cc2N(CC(=O)O)CC(=O)O)c(N(CC(=O)[O-])CC(=O)O)c1]

Unlike previously disclosed triggering mechanisms, the cation-triggered liposomes disclosed herein are capable of rapidly releasing large macromolecules entrapped within a lipid bilayer vesicle. In one embodiment, the liposome further comprises a therapeutic agent entrapped in the lipid bilayer or within the lumen of the liposome. Advantageously, the timing of release of the agent from the lipid vesicle may be controlled as well as the location of release by timing and localizing the exposure to ionizing radiation exposure.

In accordance with one embodiment, a cation-triggered liposome is provided wherein the lipid bilayer of the liposome comprises a cation responsive membrane permeabilizing lipid and the liposome entraps an aqueous core solution comprising, a cation sequestering agent that releases cations upon exposure to radiation, and a nanoscintillator capable of emitting UV light when exposed to X rays. In a further embodiment the cation-triggered liposome also comprises a therapeutic agent entrapped in the lipid bilayer or within the lumen of the liposome. The liposome membrane of the cation triggered liposomes of the present invention can be formed using standard techniques and standard liposome-forming lipids known to those skilled in the art, including for example, the use of phospholipids selected from the group consisting of phospatidylcholine, phosphatidylethanolamine, phosphatidic acid, phospatidylglycerol, phospatidylserine, phosphatidylinositol and 1,2-distearoyl-sn-glycero-3-phosphocholine (DSPC).

In one embodiment the cation-triggered liposome comprises a therapeutic agent entrapped in the liposome, either embedded in the lipid bilayer or located in the aqueous lumen of the liposome. Examples of therapeutic agents include, but are not limited to, chemotherapeutics, biological response modifiers, biological cofactors, pharmaceuticals and radiopharmaceuticals, cell toxins, and radiation sensitizers. In one embodiment the entrapped therapeutic is a chemotherapeutic agent, an antibody, a toxin, or any combination thereof. In one embodiment the therapeutic encapsulated by the cation-triggered liposome is an anticancer agent, including for example, a chemotherapeutic agent or an immunotherapeutic composition.

The cation-triggered liposome may have a diameter within the range of about 50 to 250 nm and optionally may include a targeting molecule on the external surface of the cation-triggered liposome. The present disclosure also encompasses pharmaceutical compositions comprising any of the cation triggered liposomes of the present disclosure and a pharmaceutically acceptable carrier.

In accordance with one embodiment a method of targeted delivery of a therapeutic agent is provided wherein the therapeutic agent is entrapped by a cation triggered liposome of the present invention. In one embodiment, pharmaceutical composition comprising a cation triggered liposome of the present invention is administered parenterally, i.e., intraarterially, intravenously, intraperitoneally, subcutaneously, or intramuscularly or via aerosol. Aerosol administration methods include intranasal and pulmonary administration. More preferably, the pharmaceutical compositions are administered intravenously or intraperitoneally by a bolus injection. In one embodiment the method comprises the steps of administering to a patient in need of therapy, a composition comprising a cation triggered liposome of the present disclosure, wherein the administration is via intravenous administration. After passage of a sufficient amount of time to allow the administered cation triggered liposomes to become distributed throughout the body or concentrated at the target tissue, the target tissue is irradiated with ionizing radiation sufficient to affect activation of the cation responsive membrane permeabilizing lipid and cause the release of the lipid contents. The cation triggered liposomes accumulate in the target tissues either passively or can be targeted to the desired location using techniques known to those skilled in the art. In one embodiment a targeting ligand is covalently linked to the external surface of the liposome to enhance concentration of the administered liposome to cells or tissues that express a surface molecule that binds to the targeting ligand.

In one embodiment a method of enhancing ionizing radiation therapy in the treatment of cancer in a human patient is provided. The method comprises administering to the patient a cation triggered liposome of the present invention, optionally via intravenous administration, wherein said liposome entraps an anti-cancer therapeutic; administering the ionizing radiation therapy to target a tumor site of the patient; thereby inducing the molecular cage to release free cations into the liposome core for contact and activation of the cation responsive membrane permeabilizing lipid, wherein said activated cation responsive membrane permeabilizing lipid destabilizes the membrane of the liposome and releases the anti-cancer therapeutic at the site targeted by said ionizing radiation thus complementing the therapeutic radiation therapy and enhancing the efficacy of the ionizing radiation therapy.

In accordance with one embodiment a method of targeted delivery of a therapeutic agent to a patient is provided, wherein prior to, or subsequently to the administration of the of the cation triggered liposome to the patient, the area targeted for delivery of the therapeutic agent is seeded with radioactive material. In accordance with one embodiment the cation triggered liposomes are administered in conjunction with brachytherapy wherein the cation triggered liposome comprises an anti-cancer therapeutic that is released only in proximity to the implanted radioactive seeds, ribbons, or capsules, In one embodiment, a method of treating cancer, optionally prostate cancer, is provided wherein a radiation source is placed in sufficient proximity of a solid tumor to have a therapeutic effect on the tumor and the patient is administered cation triggered liposomes of the present disclosure that comprises a cation molecular cage that releases a cation upon exposure to UV light and a nanoscintillator that emits UV light upon exposure to radiation. In one embodiment the cation triggered liposomes are administered intravenously. In one embodiment the cation triggered liposomes are injected in proximity to the solid tumor. The cation triggered liposomes can be administered to supplement any of the types of brachytherapy, including for example, the use of low-dose rate (LDR) implants, high-dose rate (HDR) implants and permanent implants.

In one embodiment the liposome-forming lipid component of the cation triggered liposome comprises one or more standard lipids known to those skilled in the art for preparing liposomes. For example such stable liposome-forming lipids include, but are not limited to, phosphatidylcholine (PC), phosphatidylethanolamine (PE), phosphatidic acid (PA), phosphatidylglycerol (PG), phospatidylserine PS) and phosphatidylinositol (PI), and non-natural lipid(s) and cation lipid(s) such as DOTMA (N-(1-(2,3-dioxyloxy)propyl)-N,N,N-trimethyl ammonium chloride) and 1,2-distearoyl-sn-glycero-3-phosphocholine (DSPC). In one embodiment the liposomes of the present disclosure further comprise a sterol component, including for example cholesterol or known derivatives thereof.

In a further embodiment, a kit is provided for preparing cation triggered liposomes of the present disclosure. The kit comprises a stable liposome forming lipid (e.g., a phospholipid), a cation responsive membrane permeabilizing lipid and a molecular cage, that upon exposure to radiation releases cations bound to the molecular cage, and a nanoscintillator. In one embodiment the kit comprises cation responsive membrane permeabilizing lipid having the structure of compound 1, the nanoscintillator: LuPO₄ and either DM-Nitrophen™ or Bis(acetoxymethyl) 3,12-bis(2-(acetoxymethoxy)-2-oxoethyl)-5-(4,5-dimethoxy-2-nitrophenyl)-6,9-dioxa-3,12-diazatetradecane-1,14-dioate (DMNPE-4 AM-caged-calcium) as the Ca²⁺ molecular cage.

### BRIEF DESCRIPTION OF THE DRAWINGS

FIG. 1 provides the structure of typical lipid bilayer components 1,2-distearoyl-sn-glycero-3-phosphocholine (DSPC;), Cholesterol (Chol), and Sphingomyelin (SM).

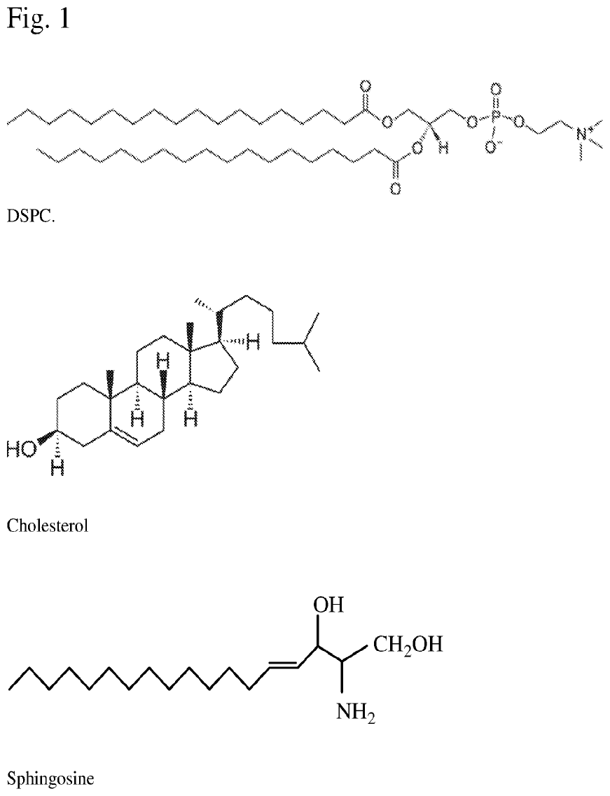

### DETAILED DESCRIPTION

### Definitions

In describing and claiming the invention, the following terminology will be used in accordance with the definitions set forth below.

The term “about” as used herein means greater or lesser than the value or range of values stated by 10 percent, but is not intended to designate any value or range of values to only this broader definition. Each value or range of values preceded by the term “about” is also intended to encompass the embodiment of the stated absolute value or range of values.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein the term “pharmaceutically acceptable salt” refers to salts of compounds that retain the biological activity of the parent compound, and which are not biologically or otherwise undesirable. Many of the compounds disclosed herein are capable of forming acid and/or base salts by virtue of the presence of amino and/or carboxyl groups or groups similar thereto.

As used herein, the term “treating” includes alleviation of the symptoms associated with a specific disorder or condition and/or preventing or eliminating said symptoms. For example, as used herein the term “treating cancer” will refer in general to reducing tumor size or preventing further growth or spread of neoplastic tissues as well as elimination of detectable cancer cells.

As used herein an “effective” amount or a “therapeutically effective amount” of a therapeutic agent refers to a sufficient amount of the agent to provide the desired effect. The amount that is “effective” will vary from subject to subject, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

The term, “parenteral” means not through the alimentary canal but by some other route such as subcutaneous, intramuscular, intraspinal, or intravenous.

As used herein, the term “purified” and like terms relate to the isolation of a molecule or compound in a form that is substantially free of contaminants normally associated with the molecule or compound in a native or natural environment.

As used herein, the term “purified” does not require absolute purity; rather, it is intended as a relative definition. The term “purified polypeptide” is used herein to describe a polypeptide which has been separated from other compounds including, but not limited to nucleic acid molecules, lipids and carbohydrates.

The term “isolated” requires that the referenced material be removed from its original environment (e.g., the natural environment if it is naturally occurring). For example, a naturally-occurring polynucleotide present in a living animal is not isolated, but the same polynucleotide, separated from some or all of the coexisting materials in the natural system, is isolated.

As used herein, the term “peptide” encompasses a sequence of 3 or more amino acids and typically less than 50 amino acids, wherein the amino acids are naturally occurring or non-naturally occurring amino acids. Non-naturally occurring amino acids refer to amino acids that do not naturally occur in vivo but which, nevertheless, can be incorporated into the peptide structures described herein.

As used herein, the terms “polypeptide” and “protein” are terms that are used interchangeably to refer to a polymer of amino acids, without regard to the length of the polymer. Typically, polypeptides and proteins have a polymer length that is greater than that of “peptides.”

As used herein an amino acid “substitution” refers to the replacement of one amino acid residue by a different amino acid residue.

As used herein, the term “conservative amino acid substitution” is defined herein as exchanges within one of the following five groups:
I. Small aliphatic, nonpolar or slightly polar residues:
   Ala, Ser, Thr, Pro, Gly;
II. Polar, negatively charged residues and their amides and esters:
   Asp, Asn, Glu, Gln, cysteic acid and homocysteic acid;
III. Polar, positively charged residues:
   His, Arg, Lys; Ornithine (Orn)
IV. Large, aliphatic, nonpolar residues:
   Met, Leu, Ile, Val, Cys, Norleucine (Nle), homocysteine
V. Large, aromatic residues:
   Phe, Tyr, Trp, acetyl phenylalanine

As used herein a general reference to a peptide is intended to encompass peptides that have modified amino and carboxy termini. For example, an amino acid chain comprising an amide group in place of the terminal carboxylic acid is intended to be encompassed by an amino acid sequence designating the standard amino acids.

As used herein, the term “alkyl” refers to a linear or branched hydrocarbon containing the indicated number of carbon atoms. Exemplary alkyls include methyl, ethyl, and linear propyl groups. The designation C₁-Cₙ alkyl is an abbreviation designating an alkyl chain having one to “n” carbons in the hydrocarbon chain.

As used herein, the term “heteroalkyl” refers to a linear or branched hydrocarbon containing the indicated number of carbon atoms and at least one heteroatom in the backbone of the structure. Suitable heteroatoms for purposes herein include but are not limited to N, S, and O.

As used herein the term “cation responsive membrane permeabilizing lipid” defines a lipid that when present as part of a lipid bilayer and in the absence of cations lacks significant synthetic lipid membrane permeabilization activity, but upon contact with cations such as Ca²⁺, will undergo a conformation change that causes the formation of macromolecule-sized defects in synthetic lipid membranes.

As used herein the term “cation molecular cage” is a chemical structure having a three dimensional space surrounded by anions that holds one or more cations within said space. A “releaseable cation molecular cage” is a cation molecular cage that upon contact with an activating agent will release the entrapped cation from the molecular cage.

As used herein the term “radiation” or “radiation source” encompasses the emission of energy as electromagnetic waves or as moving subatomic particles, especially high-energy particles that cause ionization. Examples of radiation include but are not limited to UV rays, X-rays, beta particles, and gamma rays.

The term “lipid”, as referred to herein, means a long-chain molecule comprised of fatty acids that may form liposomes under suitable liposome forming conditions. Examples of such lipids include, but are not limited to, phosphatidylcholine (PC), phosphatidylethanolamine (PE), phosphatidic acid (PA), phosphatidylglycerol (PG), and non-natural lipid(s) and cation lipid(s) such as DOTMA (N-(1-(2,3-dioxyloxy)propyl)-N,N,N-trimethyl ammonium chloride) and 1,2-distearoyl-sn-glycero-3-phosphocholine (DSPC).

The term “liposome” refers to a microscopic vesicle comprising a lipid bilayer that forms the vesicle and defines an interior enclosed luminal space of the liposome. Structurally, liposomes range in size and shape from long tubes to spheres and can be as small as 25 nm and as large as 500 nm in diameter.

### Embodiments

In accordance with one embodiment a cation triggered liposome is provided wherein said liposome comprises a liposome-forming lipid, a cation responsive membrane permeabilizing lipid; and a cation molecular cage. In one embodiment the liposome further comprises a nanoscintillator suspended in an aqueous solution encapsulated within said liposome. In one embodiment the aqueous solution further comprises a therapeutic agent that is released upon exposure of the liposome to ionizing radiation. In one embodiment the liposome comprises a therapeutic agent entrapped within the lipid bilayer. In accordance with one embodiment a cation triggered liposome is formed comprising a lipid bilayer membrane that defines and aqueous internal space, wherein said lipid bilayer membrane comprises a liposome-forming lipid and a cation responsive membrane permeabilizing lipid, and said aqueous internal space comprises a cation molecular cage. In one embodiment the liposome comprises a therapeutic agent that upon activation of the releaseable cation molecular cage entrapped by the liposome, the released cations will induce a conformational change in said cation responsive membrane permeabilizing lipid resulting in the disruption of the liposomal membrane and increased release of therapeutic compounds entrapped by the liposome.

In one embodiment, the cation triggered liposome further comprises a nanoscintillator and the releaseable cation molecular cage is designed to release cations upon stimulation by UV light. In this embodiment, upon exposure of the liposome to ionizing radiation, the nanoscintillator present in the liposomes will emit UV light which will induce the cation molecular cage to release free cations into the core aqueous solution of the liposome for contact with the cation responsive membrane permeabilizing lipids and induction of a conformational change in the cation responsive membrane permeabilizing lipids. The induced conformation change in the cation responsive membrane permeabilizing lipids destabilizes the lipid membrane of the liposome resulting in the release of the liposomal contents.

In accordance with one embodiment the cation triggered liposomes of the present disclosure are small uni-lamellar vesicles (SUV's) ranging in size from about 50 to about 250 nm in diameter. In one embodiment the liposomes have a diameter within the range of about 100 to 200 nm.

In one embodiment the stable lipids forming the liposomal membrane are phospholipids selected from the group consisting of phospatidylcholine, phosphatidylethanolamine, phosphatidic acid, phospatidylglycerol, phospatidylserine, phosphatidylinositol and 1,2-distearoyl-sn-glycero-3-phosphocholine (DSPC). In one embodiment the stable lipids forming the liposomal membrane consist of phospatidylcholine and phosphatidylethanolamine. In one embodiment the liposomal membrane further comprises cholesterol. Such lipids are combined with a cation responsive membrane permeabilizing lipid to form the cation triggered liposomes of the present disclosure.

In one embodiment the stable liposome-forming lipids include, but are not limited to, phosphatidylcholine (PC), phosphatidylethanolamine (PE), phosphatidic acid (PA), phosphatidylglycerol (PG), phospatidylserine PS) and phosphatidylinositol (PI), and non-natural lipid(s) and cation lipid(s) such as DOTMA (N-(1-(2,3-dioxyloxy)propyl)-N,N,N-trimethyl ammonium chloride), distearoylPC, bis-SorbPC17,17, and 1,2-distearoyl-sn-glycero-3-phosphocholine (DSPC).

In one embodiment the liposome comprises one or more stable lipids selected from the group consisting of 1,2 dioleoyl-3-trimethylammonium-propane DOTAP, dioctadecyldimethylammonium chloride DODAc, 1,2-dimyristoyloxypropyl-3-dimethyl-hydroxyethyl ammonium DMRIE, 2,3-dioleoyloxy-N-(2(sperminecarboxamide)ethyl)-N,N-dimethyl-1 propananninium DOSPA, 1,2-dimethyl-dioctadecylammoniumbromide DDAB, 2-dioleyl-3-N,N,N-trimethylaminopropanechloride DOTMA, 1,2-dimyristoyl-3-trimethylammoniumpropane DMTAP, 1,2-distearoyl-3-trimethylammoniumpropane DSTAP, 1,2-Dioleoyl-3-dimethylammonium-propane DODAP, 1,2-dioleoyl-sn-glycero-3-phosphoethanolamine DOPE and N-(4-carboxybenzyl)-N,N-dimethyl-2,3-bis(oleoyloxy)propan-1-aminium DOBAQ and dioctadecylamidoglycylspermine DOGS.

In one alternative embodiment, any of the cation triggered liposomes disclosed herein can be further modified to enhance their stability prior to being subjected to radiation. In one embodiment the cation triggered liposomes are stabilized by coating their surface with a “stealth” material such as polyethylene glycol (PEG). PEGylated liposomes can not only prevent liposomes from fusing with one another but also enhance their in vivo circulation lifetime by suppressing plasma proteins from adsorbing onto the liposome. PEG can be covalently linked to the polar head groups of the phospholipids comprising the liposome using standard techniques known to those skilled in the art. Examples of PEG liposome compositions are various combinations of PEG and PCs, and/or PEs, and/or PAs, and/or PGs, and/or sterols such as cholesterol, and/or non-natural lipids, and/or cation lipids. In one embodiment of the present disclosure, the liposome(s) are comprised of PEG₂₀₀₀-dioleoylPE, cholesterol, dioleoylPC, and bis-SorbPC_{17,17}. In another embodiment, the liposome(s) are comprised of PEG₂₀₀₀-distearoylPE, cholesterol, distearoylPC, and bis-SorbPC_{17,17}. In yet another embodiment, the liposome(s) are comprised of PEG₂₀₀₀-distearoylPE, distearoylPC.

In accordance with one embodiment, any of the cation triggered liposomes disclosed herein can further comprise a therapeutic agent entrapped in the liposome, either embedded in the lipid bilayer or located in the aqueous lumen of the liposome. Examples of therapeutic agents include, but are not limited to, chemotherapeutics, biological response modifiers, biological cofactors, pharmaceuticals and radiopharmaceuticals, cell toxins, radiation sensitizers, and genetic materials. In one embodiment the entrapped therapeutic is a chemotherapeutic agent, an antibody, a toxin, or any combination thereof.

The present disclosure also encompasses pharmaceutical compositions comprising any of the cation triggered liposomes of the present disclosure and a pharmaceutically acceptable carrier. The pharmaceutical composition may include or be associated with an additional suitable pharmaceutical carrier or diluent. The releasable agent entrapped by the liposome may be a therapeutic or diagnostic agent. Carrying a therapeutic or diagnostic agent within or associated with a liposome provides for a biocompatible and non-toxic means of in vivo delivery. Chemotherapeutics, biological response modifiers, biological cofactors, pharmaceuticals and radiopharmaceuticals, cell toxins, radiation sensitizers, genetic materials, contrast agents, iodinated agents, fluorescent compounds, agents containing MRS/MRI sensitive nuclides, and the like, may be encapsulated in or associated with the liposomes of the present disclosure and released at desired target sites.

In another embodiment, a method of treating a condition responsive to a liposome-encapsulated or associated therapeutic agent is provided. The method comprises the steps of (i) administering to a patient a pharmaceutical composition comprising a cation triggered liposomal delivery system comprising a therapeutic agent, wherein the therapeutic agent is encapsulated in or associated with the liposome, and a pharmaceutically acceptable carrier or diluent; and (ii) subjecting the patient to radiation in order to destabilize the liposome and release the therapeutic agent encapsulated in or associated with the liposome. In one embodiment, the radiation dosage ranges from about 5 to about 500 rads. In one embodiment, the radiation dosage ranges from about 50 to about 250 rads. Examples of therapeutic agents include, but are not limited to, chemotherapeutics, biological response modifiers, biological cofactors, pharmaceuticals and radiopharmaceuticals, cell toxins, radiation sensitizers and nucleic acids. Examples of conditions that are responsive to liposome-encapsulated or associated therapeutic agent(s) include, but are not limited to, cancer, immune disorders, developmental disorders, and genetic disorders.

Tumors represent a specific tissue site of considerable therapeutic interest; several research groups have reported the increased localization of sterically stabilized liposomes (PEG-liposomes) at tumor sites. The increased permeability of the vasculature at tumor sites (due to angiogenic factors secreted by tumors) allows liposomes to escape the capillaries to reach the tumor interstitial space. Sterically stabilized liposomes are more likely to accumulate at these sites because of their sustained concentration in the blood. Furthermore, it is known that the hydrophilic surface polymer may facilitate the transit from the capillaries to the tumor site. Reports of passive targeting of PEG-liposomes to tumors, including murine colon carcinomas, murine lymphomas, murine mammary carcinomas, human squamous cell lung carcinomas in SCID mice are known in the art. Specific targeting via antibodies coupled to liposomes has been observed as well. Antibody (mAb) conjugated sterically stabilized liposomes are known to localize at squamous cell carcinomas of the lung in mice and effectively deliver doxorubicin to these sites. Although the coupling of mAbs to conventional liposomes appears to increase their rate of clearance from the blood stream, the mAb conjugated PEG-liposomes, remain in circulation long enough to accumulate at their target cells. Accordingly, in one embodiment the cation triggered liposomes comprise an anti-cancer agent, including for example a chemotherapeutic agent or an immunotherpeuic composition.

The present disclosure further contemplates a targetable liposomal delivery system, wherein said system comprises a cation triggered liposome of the present invention with a therapeutic agent entrapped in said liposome, further wherein the liposome is targeted to a tumor site through attachment of at least one peptide to the exterior of the liposome. Peptides that target liposomes to tumor sites include, but are not limited to, peptide sequences, peptide fragments, antibodies, antibody fragments, and antigens.

In one embodiment a method of localized delivery of a therapeutic agent is provided. The method comprises the steps of administering to a patient in need of therapy, a pharmaceutical composition comprising a cation triggered liposome of the present disclosure. Preferably, the pharmaceutical compositions are administered parenterally, i.e., intraarterially, intravenously, intraperitoneally, subcutaneously, or intramuscularly or via aerosol. Aerosol administration methods include intranasal and pulmonary administration. More preferably, the pharmaceutical compositions are administered intravenously or intraperitoneally by a bolus injection. After passage of a sufficient amount of time to allow the administered cation triggered liposomes to become associated with the target tissue, the target tissue is irradiated with ionizing radiation sufficient to affect release of free cations from the cation molecular cage to induce a conformational change in the cation responsive membrane permeabilizing lipid resulting in the release of the lipid contents. The cation triggered liposomes can accumulate in the target tissues either passively or can be targeted to the desired location using techniques known to those skilled in the art.

Due to the ability to locally deliver chemotherapeutics to tumor cells, the presently disclosed cation sensitive liposomes can be used to simultaneously administer radiation treatment and chemotherapeutic treatment to a cancer patient in need thereof. In accordance with one embodiment a method of enhancing ionizing radiation therapy in the treatment of cancer in a human patient is provided. The method comprises administering to the patient a cation triggered liposome, wherein said liposome entraps an anti-cancer therapeutic, including for example a chemotherapeutic or cytotoxic agent. The tumor site of the patient is then subjected to a therapeutic dose of ionizing radiation therapy that simultaneously destabilizes the membrane of the cation triggered liposomes causing them to release the anti-cancer therapeutic at the targeted site to complement the therapeutic radiation therapy and enhance the efficacy of the ionizing radiation therapy.

In another embodiment of the present disclosure a method of producing a cation sensitive liposomes is provided. The method encompasses drying the lipids that comprise the liposomes, hydrating the lipids with a buffer comprising agents to be encapsulated or associated in a desired molar ratio to create hydrated bilayers, converting the bilayers into liposomes, and purifying the liposomes. In one embodiment the lipids are dried in an oxygen free environment, such as an argon stream, and the bilayers are converted into liposomes by ultrasonification or freeze-thawing-extrusion. The liposomes may be purified with gel permeation chromatography or other methods.

### Exemplified Embodiments

In accordance with embodiment 1, a cation triggered liposome is provided, wherein the liposome comprises
a lipid bilayer that comprises a liposome-forming lipid and a cation responsive membrane permeabilizing lipid;
a molecular cage that reversibly binds a cation and releases bound cations upon activation; and
a therapeutic agent entrapped in the lumen or within the lipid bilayer of the liposome.

In accordance with embodiment 2, a cation triggered liposome of embodiment 1 is provided wherein the liposome further comprises a nanoscintillator that emits UV light upon exposure to radiation, wherein said molecular cage releases cations upon exposure to UV light.

In accordance with embodiment 3, a cation triggered liposome of embodiment 2 is provided, wherein said nanoscintillator emits UV light upon exposure to X-rays.

In accordance with embodiment 4, a cation triggered liposome of any one of embodiments 1-3 is provided wherein said liposome comprises an aqueous core encapsulated within said liposome, said aqueous core comprising said nanoscintillator and said molecular cage.

In accordance with embodiment 5, a cation triggered liposome of any one of embodiments 1-4 is provided further comprising a therapeutic agent entrapped in the lipid bilayer or within the lumen of the liposome.

In accordance with embodiment 6, a cation triggered liposome of any one of embodiments 1-5 is provided wherein the liposome comprising polyethylene glycol polymers attached to the exterior surface of the liposome.

In accordance with embodiment 7, a cation triggered liposome of any one of embodiments 1-6 is provided wherein the cation responsive permeabilizing lipid has the general structure of

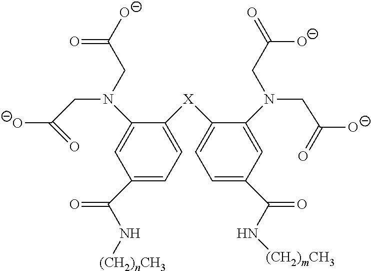

wherein X is a linker of 3 to 6 atoms selected from C₁-C₆ alkyl and C₁-C₅ heteroalkyl, optionally wherein X is —O(CH₂)_{z}O—, where z is an integer selected from 1 to 4; and
n and m are independently an integer selected from the range of 9-17.
   In accordance with embodiment 8, a cation triggered liposome of embodiment 7 is provided wherein the cation responsive permeabilizing lipid has the general structure of compound 1:

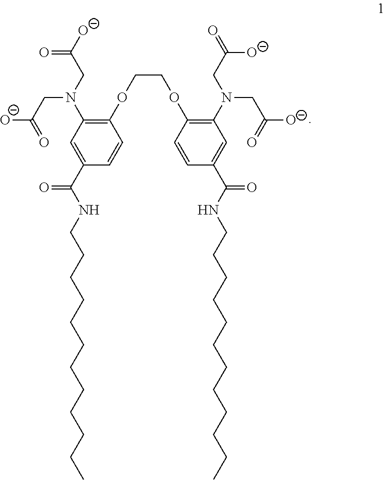 [SMILES: CCCCCCCCCCCCNC(=O)c1ccc(OCCOc2ccc(C(=O)NCCCCCCCCCCCC)cc2N(CC(=O)[O-])CC(=O)[O-])c(N(CC(=O)[O-])CC(=O)[O-])c1]

In accordance with embodiment 9, a cation triggered liposome of any one of embodiments 1-8 is provided wherein said molecular cage is DM-Nitrophen™.

In accordance with embodiment 10, a cation triggered liposome of any one of embodiments 1-9 is provided wherein the nanoscintillator is LuPO₄

In accordance with embodiment 11, a cation triggered liposome of any one of embodiments 1-10 is provided wherein said liposome-forming lipid is a phospholipid selected from the group consisting of phospatidylcholine, phosphatidylethanolamine, phosphatidic acid, phospatidylglycerol, phospatidylserine, phosphatidylinositol and 1,2-distearoyl-sn-glycero-3-phosphocholine (DSPC).

In accordance with embodiment 12, a cation triggered liposome of any one of embodiments 1-11 is provided wherein the liposome further comprises cholesterol.

In accordance with embodiment 13, a cation triggered liposome of any one of embodiments 1-12 is provided further comprising an anticancer agent entrapped by said liposome.

In accordance with embodiment 14, a cation triggered liposome of embodiment 13 is provided wherein the anticancer agent is a chemotherapeutic agent or an immunotherapeutic composition.

In accordance with embodiment 15, a cation triggered liposome of any one of embodiments 1-14 is provided wherein said liposome has a diameter within the range of about 50 to 250 nm.

In accordance with embodiment 16, a cation triggered liposome of any one of embodiments 1-15 is provided wherein the liposome further comprises a targeting molecule on the external surface of the liposome.

In accordance with embodiment 17, a cation triggered liposome of embodiments 16 is provided wherein the targeting molecule is a ligand that specifically binds to a surface molecule present on a solid tumor.

In accordance with embodiment 18, a cation triggered liposome of any one of embodiments 1-17 is provided wherein the cation responsive membrane permeabilizing lipids are present at 5, 6, 7, 8, 9, 10 or 20% relative to the total phospholipids comprising the liposome bilayer.

In accordance with embodiment 19, a cation triggered liposome of any one of embodiments 1-18 is provided wherein the liposome bilayer further comprises cholesterol.

In accordance with embodiment 20, a method of enhancing ionizing radiation therapy in the treatment of cancer in a human patient is provided, wherein the method comprises:
i) administering to the patient a cation triggered liposome of any one of embodiments 1-19, wherein said liposome entraps an anti-cancer therapeutic;
ii) administering the ionizing radiation therapy to target a tumor site of the patient; thereby inducing the molecular cage to release free cations, wherein the free cations, upon contact with the cation responsive membrane permeabilizing lipids, induce a conformational change in said cation responsive membrane permeabilizing lipid that destabilizes the membrane of the liposome to release the anti-cancer therapeutic at the site targeted by said ionizing radiation to complement the therapeutic radiation therapy and enhance the efficacy of the ionizing radiation therapy.

In accordance with embodiment 21, a method of enhancing brachytherapy in the treatment of cancer in a human patient is provided, wherein the method comprises:
administering to the patient a cation triggered liposome of any one of embodiments 1-19, wherein said liposome entraps an anti-cancer therapeutic;
implanting a radiation source within sufficient proximity of solid tumor to have a therapeutic effect on said tumor
wherein said radiation source activates said nanoscintillator of the cation triggered liposomes at the tumor site; thereby inducing the molecular cage to release free cations, wherein the free cations, upon contact with the cation responsive membrane permeabilizing lipids, induce a conformational change in said cation responsive membrane permeabilizing lipid that destabilizes the membrane of the liposome to release the anti-cancer therapeutic at the site of the implanted radiation source to complement the therapeutic brachytherapy and enhance the efficacy of the ionizing radiation therapy.

In accordance with embodiment 22, a kit is provided for preparing cation triggered liposomes of the present disclosure, wherein the kit comprises a stable liposome forming lipid (e.g., a phospholipid), a cation responsive membrane permeabilizing lipid and a molecular cage, that upon exposure to radiation releases cations bound to the molecular cage, and a nanoscintillator. In one embodiment the kit comprises cation responsive membrane permeabilizing lipid having the structure of

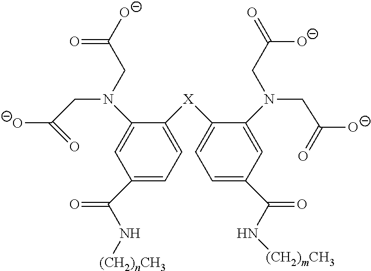

wherein X is a linker of 3 to 6 atoms selected from C₁-C₆ alkyl, C₁-C₅ heteroalkyl, and —O(CH₂)_{z}O—, where z is an integer selected from 1 to 4, optionally wherein X is —O(CH₂)_{z}O—; and the molecular cage is a Ca2+ molecular cage, optionally wherein the nanoscintillator is LuPO4 and the Ca2+ molecular cage is selected from DM-Nitrophen™ or Bis(acetoxymethyl) 3,12-bis(2-(acetoxymethoxy)-2-oxoethyl)-5-(4,5-dimethoxy-2-nitrophenyl)-6,9-dioxa-3,12-diazatetradecane-1,14-dioate (DMNPE-4 AM-caged-calcium).

What is claimed is:

1. A cation triggered liposome, said liposome comprising
a lipid bilayer that comprises a liposome-forming lipid and a cation responsive membrane permeabilizing lipid;
a molecular cage that reversibly binds a cation and releases bound cations upon activation; and
a therapeutic agent entrapped in the lumen or within the lipid bilayer of the liposome.

2. The cation triggered liposome of claim 1 further comprising a nanoscintillator that emits UV light upon exposure to radiation, wherein said molecular cage releases cations upon exposure to UV light.

3. The cation triggered liposome of claim 2, wherein said nanoscintillator emits UV light upon exposure to X-rays.

4. The cation triggered liposome of claim 3 wherein said liposome comprises
an aqueous core encapsulated within said liposome, said aqueous core comprising said nanoscintillator and said molecular cage.

5. The cation triggered liposome of claim 3 further comprising a therapeutic agent entrapped in the lipid bilayer or within the lumen of the liposome.

6. The cation triggered liposome of claim 1 wherein the cation responsive permeabilizing lipid has the general structure of 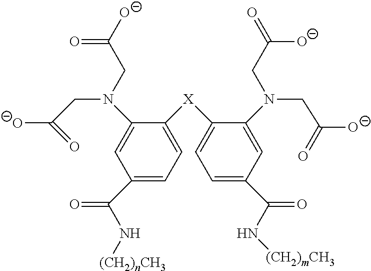
wherein X is a linker of 3 to 6 atoms selected from C₁-C₆ alkyl and C₁-C₅ heteroalkyl, optionally
wherein X is —O(CH₂)_{z}O—, where z is an integer selected from 1 to 4; and
n and m are independently an integer selected from the range of 9-17.

7. The cation triggered liposome of claim 6 wherein the cation responsive permeabilizing lipid has the general structure of compound 1:

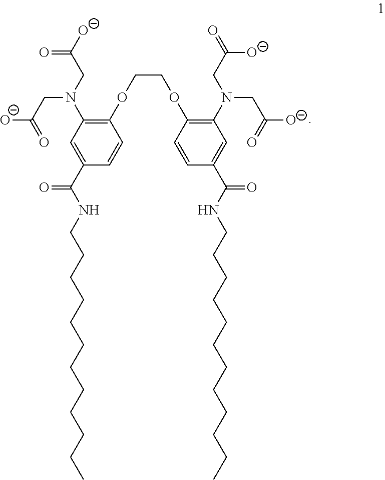 [SMILES: CCCCCCCCCCCCNC(=O)c1ccc(OCCOc2ccc(C(=O)NCCCCCCCCCCCC)cc2N(CC(=O)[O-])CC(=O)[O-])c(N(CC(=O)[O-])CC(=O)[O-])c1]

8. The cation triggered liposome of claim 7 wherein said molecular cage is DM-Nitrophen™.

9. The cation triggered liposome of claim 8 wherein the nanoscintillator is LuPO₄

10. The liposome of claim 9 wherein said liposome-forming lipid is a phospholipid selected from the group consisting of phospatidylcholine, phosphatidylethanolamine, phosphatidic acid, phospatidylglycerol, phospatidylserine, phosphatidylinositol and 1,2-distearoyl-sn-glycero-3-phosphocholine (DSPC).

11. The liposome of claim 10 wherein the liposome further comprises cholesterol.

12. The liposome of claim 1 further comprising an anticancer agent entrapped by said liposome.

13. The liposome of claim 12 wherein the anticancer agent is a chemotherapeutic agent or an immunotherapeutic composition.

14. The liposome of claim 9 wherein said liposome has a diameter within the range of about 50 to 250 nm.

15. The liposome of claim 14 wherein the liposome further comprises a targeting molecule on the external surface of the liposome.

16. A method of enhancing ionizing radiation therapy in the treatment of cancer in a human patient, said method comprising:
administering to the patient a cation triggered liposome of claim 1, wherein said liposome entraps an anti-cancer therapeutic;
administering the ionizing radiation therapy to target a tumor site of the patient; thereby inducing the molecular cage to release free cations, wherein the free cations, upon contact with the cation responsive membrane permeabilizing lipids, induce a conformational change in said cation responsive membrane permeabilizing lipid that destabilizes the membrane of the liposome to release the anti-cancer therapeutic at the site targeted by said ionizing radiation to complement the therapeutic radiation therapy and enhance the efficacy of the ionizing radiation therapy.

17. A method of enhancing brachytherapy in the treatment of cancer in a human patient, said method comprising:
administering to the patient a cation triggered liposome of claim 1, wherein said liposome entraps an anti-cancer therapeutic;
implanting a radiation source within sufficient proximity of solid tumor to have a therapeutic effect on said tumor
wherein said radiation source activates said nanoscintillator of the cation triggered liposomes at the tumor site; thereby inducing the molecular cage to release free cations, wherein the free cations, upon contact with the cation responsive membrane permeabilizing lipids, induce a conformational change in said cation responsive membrane permeabilizing lipid that destabilizes the membrane of the liposome to release the anti-cancer therapeutic at the site of the implanted radiation source to complement the therapeutic brachytherapy and enhance the efficacy of the ionizing radiation therapy.
